(19) United States
(12) Patent Application Publication
(10) Pub. No.: US 2023/0317291 A1
(43) Pub. Date: Oct. 5, 2023

(54) Clinical Contextual Insight and Decision Support Visualization Tool

(71) Applicant: Parkland Center for Clinical Innovation, Dallas, TX (US)

(72) Inventors: Manjula Julka, Plano, TX (US); Priyanka Kharat, Dallas, TX (US); Vikas Chowdhry, Southlake, TX (US); Akshay Arora, Irving, TX (US)

(21) Appl. No.: 17/666,453

(22) Filed: Feb. 7, 2022

Related U.S. Application Data
(60) Provisional application No. 63/146,564, filed on Feb. 5, 2021.

(51) Int. Cl. G16H 50/30; G16H 50/20; G16H 10/60

(52) U.S. Cl. CPC G16H 50/30; G16H 50/20; G16H 10/60

(57) ABSTRACT

A clinical insight and decision support visualization tool includes a data ingestion logic module that automatically accesses, in real-time, electronic health data of a plurality of trauma patients being treated by a care team at a healthcare institution. The tool further includes a data analysis module that automatically applies at least one predictive model to analyze the processed patient data and determine a current mortality risk score value for each patient, and a user interface module that presents real-time and historical information for each trauma patient including a current mortality risk score value, a categorization of the current mortality risk score value, top contributors to the current mortality risk score value and their respective values, and a trend plot of historic mortality risk score values.

### FIELD

The present application claims the benefit of U.S. Provisional Patent Application No. 63/146,564 filed on Feb. 5, 2021, the entirely of which is incorporated herein by reference.

### FIELD

The present disclosure relates to a system and method for clinical insight and decision support visualization tool for the care and treatment of patients, by way of example trauma patients in trauma centers.

### BACKGROUND

Treatment for polytrauma patients is difficult to manage because their condition typically involves multiple organ systems, physiological derangement, lack of historical information, and fluctuating level of consciousness upon arrival. Treatment for these patients and patients with other complex emergent conditions require quick clinical decision-making and life-saving interventions in the critical window of the first 72 hours.

### BRIEF DESCRIPTION OF THE DRAWINGS

FIG. 1 is a simplified block diagram of an embodiment of a clinical insight and decision support tool according to the teachings of the present disclosure;

FIG. 2 is a more detailed block diagram of an embodiment of a clinical insight and decision support tool according to the teachings of the present disclosure;

FIG. 3 is a more detailed block diagram of an embodiment of a clinical insight and decision support tool according to the teachings of the present disclosure;

FIGS. 4-11 are exemplary screenshots showing the types of information available from a clinical insight and decision support tool according to the teachings of the present disclosure;

FIG. 12A illustrates the configurable mortality risk of score computation window;

FIG. 12B illustrates the configurable mortality risk of score computation window with a relaxation period; and

FIG. 13 is a simplified block diagram of the computing environment of an embodiment of the clinical insight and decision support tool according to the teachings of the present disclosure.

### DETAILED DESCRIPTION

There are multiple management strategies that can be utilized for polytrauma patients including: damage control, early total care, and early appropriate care. The care team, often consisting of 15-20 people led by a trauma surgeon, must take into account all relevant physiological changes and make a variety of significant and consequential decisions in rapid. succession—when to stabilize versus intervene, how to sequence interventions, etc. In many cases, the input to the decision-making process can feel like a confluence of instinct, art, and science that relies on a foundation of clinical experience accumulated over many years. Because. of the time-critical nature of caring and treating polytrauma patients, a clinical contextual insight and decision tool 10 has been developed to assist the care team in making these life-saving decisions. This tool may be used with a predictive model to provide the relevant inputs or the tool may be customized by the end user to display relevant information and trends for an emergent condition that is not yet modeled.

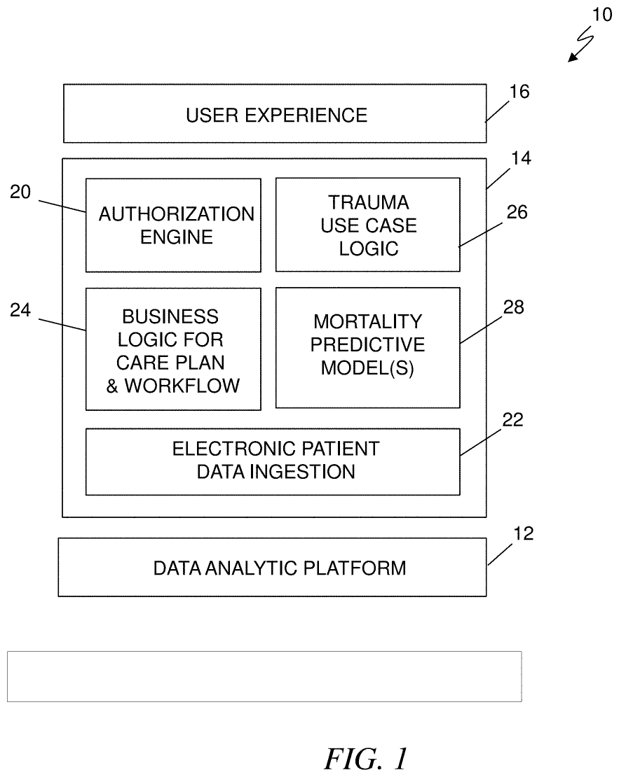

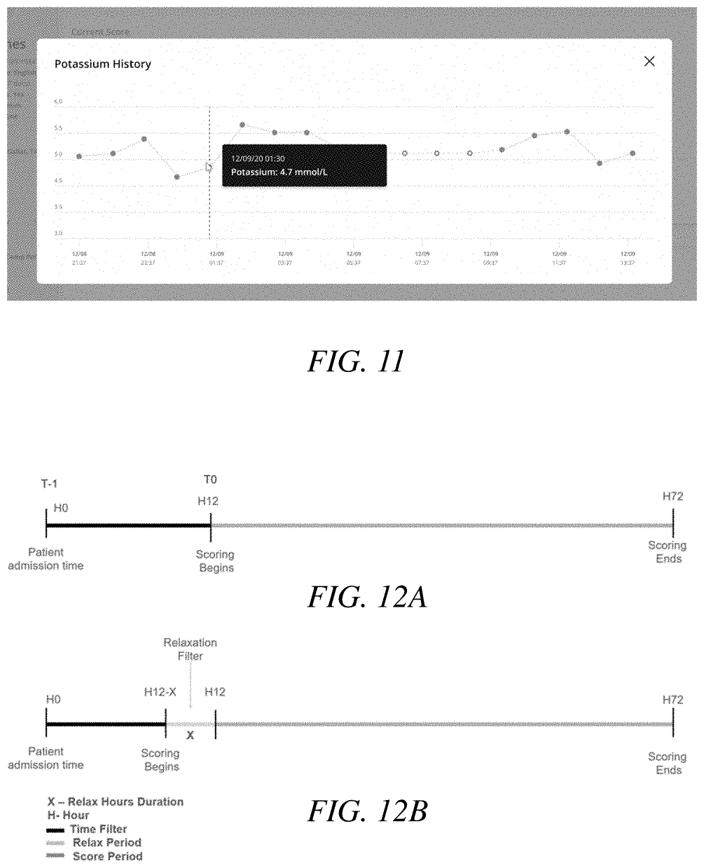

Referring to FIG. 1, the clinical contextual insight and decision support tool 10 is implemented in an electronic medical/health record (EMR or EHR) standard-agnostic application framework architecture that is preferably implemented on a cloud-based data analytic platform 12 that supports an application framework 14 and a use-case specific user experience data presentation module 16. The tool 10 includes a Fast Health Interoperability Resources (FHIR) agnostic authorization engine 20 that enables an automatic and interoperable connection 22 with EHR/EMR sources to ingest, exchange and translate complex real-time and non-real time electronic patient data. The ingested patient information may include those data needed to determine a mortality risk score, but other types of data such as case manager's notes, etc. and non-EHR data that may be used to formulate a care plan that presents data as a meaningful part of the care team's workflow in a consistent, contextual and timely manner 24. The tool 10 implements a trauma use case logic 26 and determines a risk score by using one or more predictive models 28 for mortality for polytrauma conditions (or other modeled emergent conditions or directly from the EMR/EHR for non-modeled emergent conditions based on provider selection of relevant inputs. The analytic platform 12 includes machine learning logic that fine-tunes and refines the mortality risk score computation to increase the accuracy. The user experience 16 presents the mortality risk score, care plan and other relevant actionable clinical information to the care team that shows over-time trends based on relevant clinical information. The tool IO provides analytic and decision support data that facilitates the care team to make time-critical life-saving decisions. The data presented by the tool IO includes data over-time trends based on top contributors to the risk score result and relevant clinical information (e.g., changes and trends in lab results and selected vital signs over the last selected period or time, such as hourly for 72 hours post-admission or every 1/2/4/12/24 hours). As shown in FIG. 12, the mortality risk scoring time window start time (H12-X) and end time (H72) as well as risk score computation intervals can be set by the predictive model, or set according to user, departmental, or institutional preferences. The tool presents information in a user-friendly manner with the right clinical context to reduce cognitive overload of data as an important component of clinical decision support.

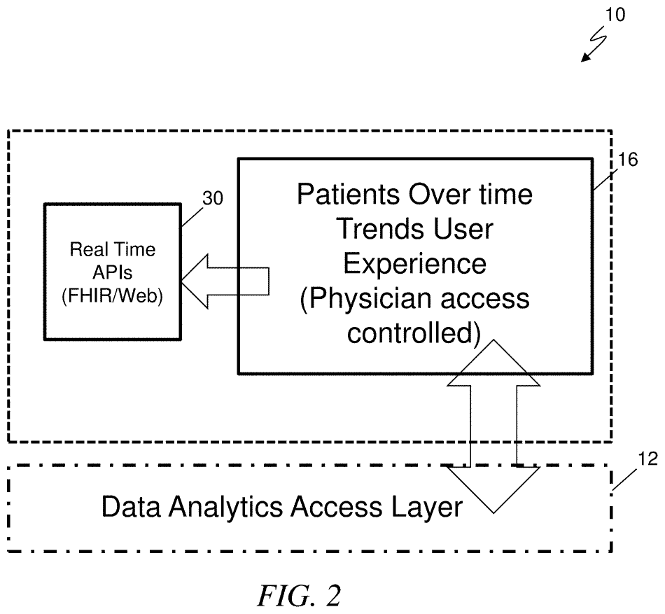

As shown in FIG. 2, the tool 10 includes real-time data integration with electronic health record (EHR) and user experience (UX) 16 integration with EHR. The tool IO is configured to compute a risk score and other complex clinical information hourly (or another desired interval) and to present the data to the user in an informative and insightful manner in real-time. The tool employs secure and robust architecture in compliance with HIPAA and/or HITRUST standards. In addition to EHR data, the tool may also ingest other non-EHR data, such as case manager's notes, the patient's care plan, etc. The tool may incorporate industry-standard “SMART-on-FHIR” methodology, and can be scaled to a multitude of EHR systems. The tool can be hosted securely on existing technology platforms with customizable database hooks to draw in a minimum set of critical information, analyze the data, and present information that assists the care team in a clinician-friendly manner. Clinical data is automatically pulled from EHR and other sources via real-time APis 30 on a regular basis to ensure that the current data is the most up to date. The data analysis interval to compute a mortality risk score may vary and be automatically adjusted, for example, the time interval may be more frequent when the patient is newly admitted and less frequently after the patient's condition becomes less critical. Alternatively, a mortality risk score is computed in real time whenever new patient data is available. Data sources may include the patient's vitals, lab results, medications, care plan, case manager's notes, Social Determinants of Health data (when available), and other data that reflect the real-time condition of the patient. It is contemplated that the data ingestion process may also ingest historical or non-real-time patient clinical and non-clinical data related to the patients if available and deemed relevant to the patient's condition.

One of the key data points in the decision-making process of picking the right strategy is to determine the risk of mortality for the patient. Traditional risk scores such as TRISS, PTGS etc. are cumbersome, static, and typically done only as a one-time mortality prediction. In the absence of a more useful mortality risk score, trauma care givers default to using vital signs, lab values etc. for decision making. That introduces the concerns of cognitive data overload, biases and use of heuristics-based mental models in the decision-making process.

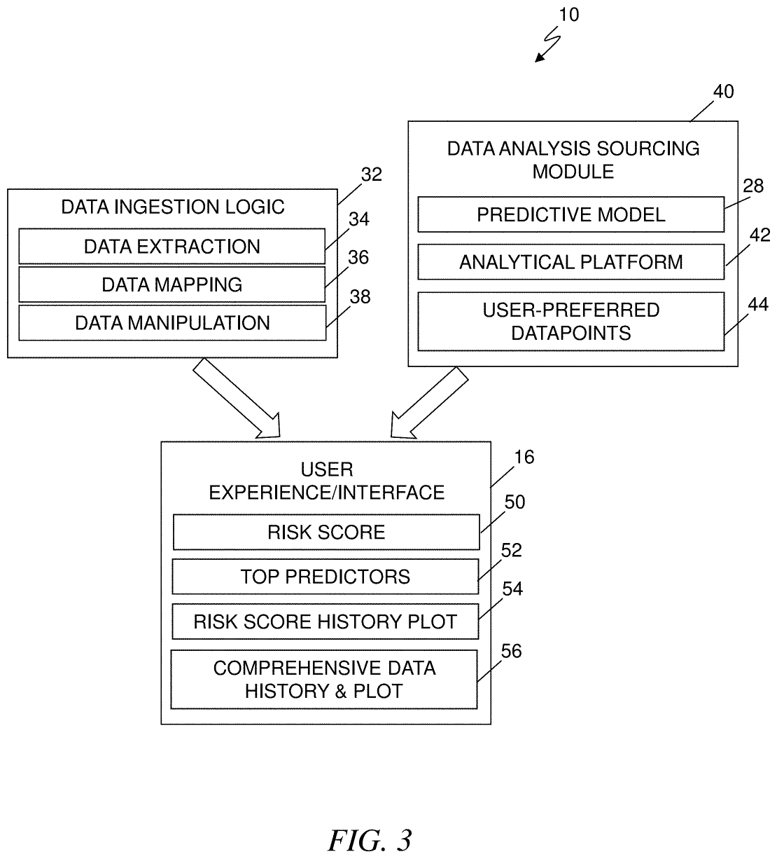

As shown in FIG. 3, the data ingestion logic 32 includes data extraction 34, data mapping, and data manipulation 38 so that the data is processed for analysis. Where there is a missing data point, extrapolation, trend analysis and other techniques may be used to determine the missing data point value. The data analysis sourcing module 40 includes one or more predictive e-models 28, other user preferred data analytics 42 and data points 44, and then presenting the information through a graphical user interface (user experience or UX) 16. The user experience/interface 16 presents the composite mortality risk score 50, the top contributors to the risk score 52, a historic plot of the risk score 54, and comprehensive patient data history and plot 56. Artificial Intelligence (AI) and Natural Language Processing (NLP) may be used to fine tune the predictive model to improve the accuracy of data analysis.

The clinical insight and decision support tool IO described herein uses a predictive model that takes into account of all available EHR and other clinical data to determine a composite mortality risk score that is indicative of the likelihood that the patient will die and shows the patient's status (risk score and clinical data) over time (trending plot). A new updated score is computed every hour or at another desired interval.

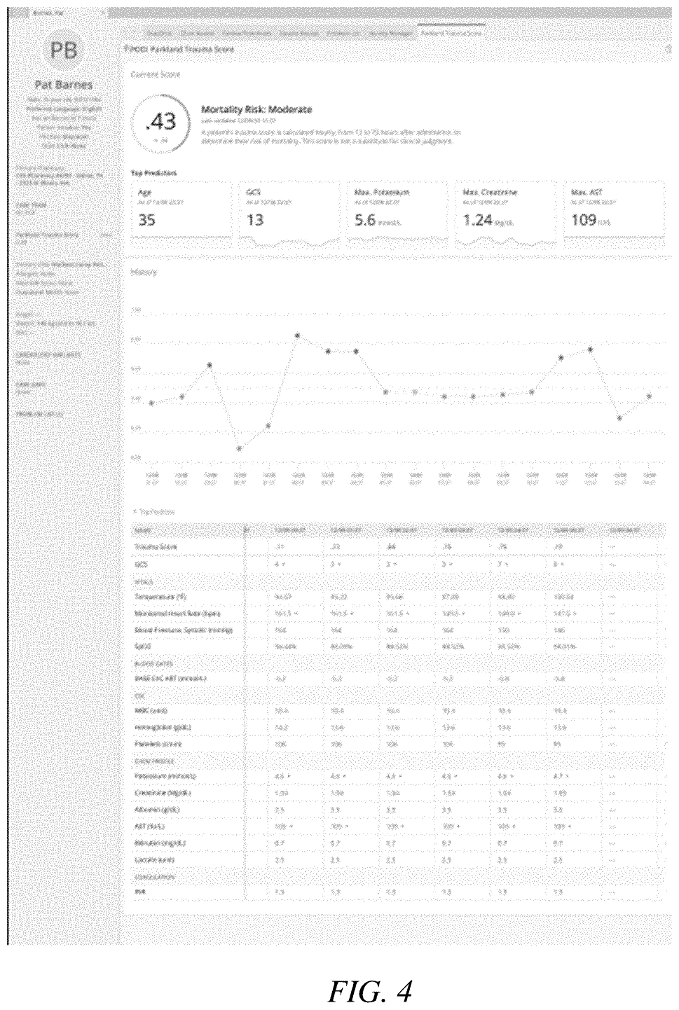

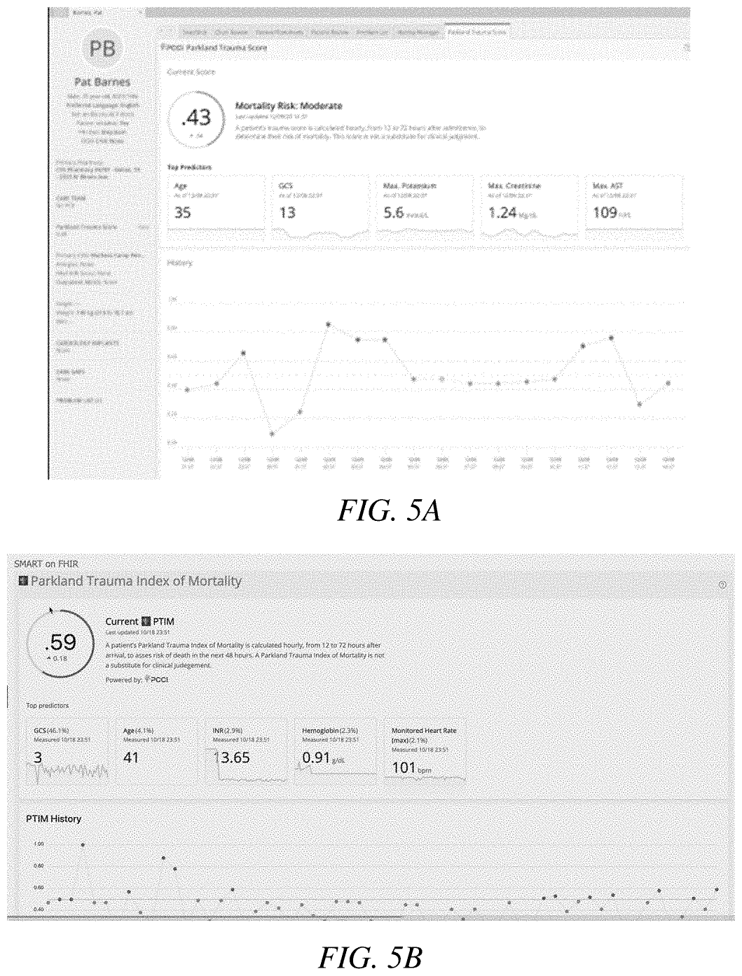

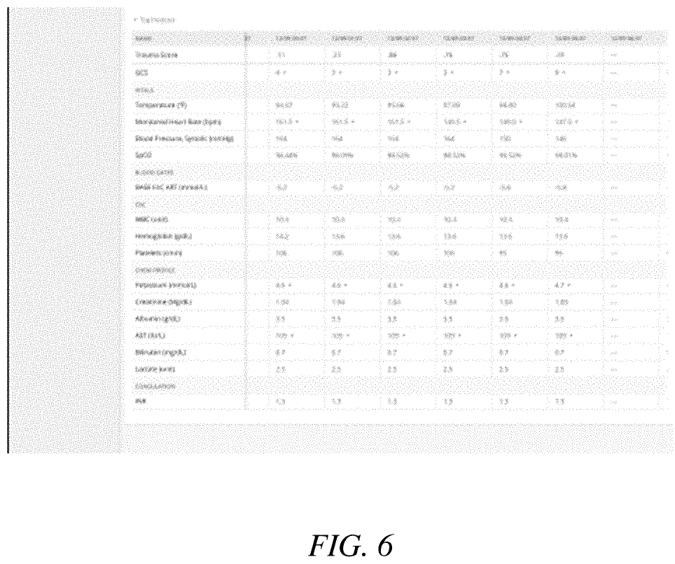

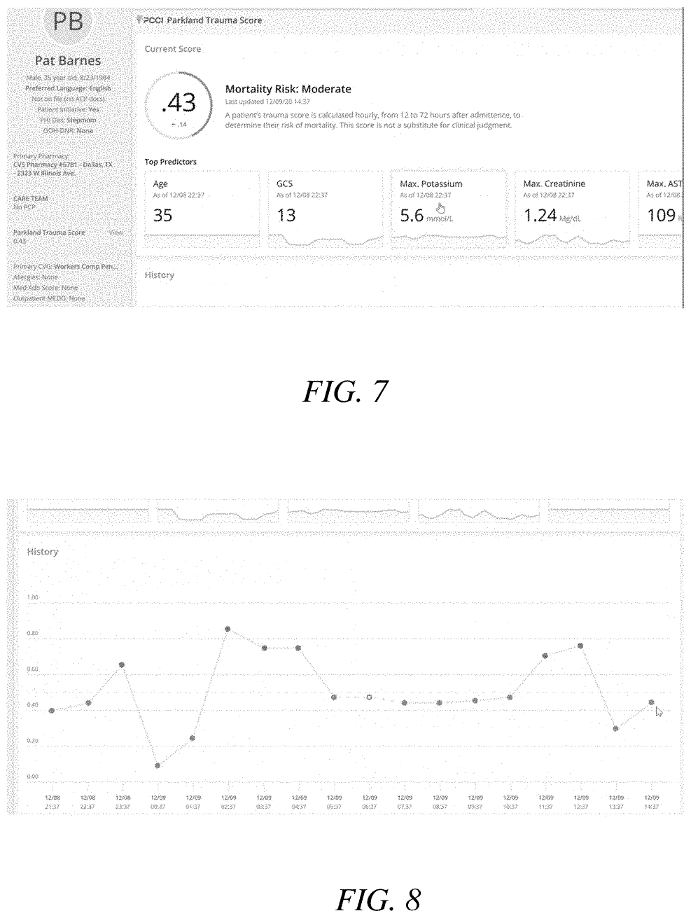

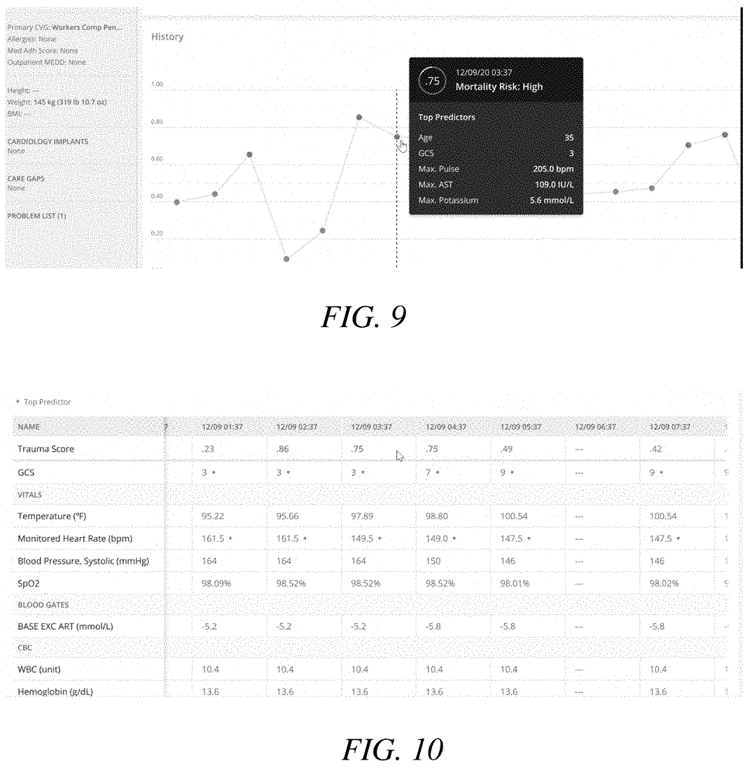

FIGS. 4-11 are exemplary screenshots showing the types of information available from the clinical insight and decision support tool IO according to the teachings of the present disclosure. The graphical user interface (user experience or UX) 16 for the system provides a mortality risk score for each patient and a risk categorization or stratification of that risk score as high, moderate, and low likelihood of death. In the example shown in the figures, the mortality risk score is 43, which is categorized as moderate risk. The user interface also provides a list of top predictors or contributors to that risk score value, their respective current values, and a historic plot. In the example shown in FIG. 7, the top predictors include age, GCS (Glasgow Coma Score/Scale indicating the patient's level of consciousness), potassium level, creatinine, and AST (aspartate aminotransferase indicative liver damage). As shown in FIG. 8, the user interface also includes a plot of historic trend of the risk score value that are color-coded to indicate risk stratification of each score (e.g., red data point meaning outside of desired range and green data point meaning inside desired range). A historic trend chart and plot are also available for each contributing factor. When the user places the pointer or cursor on any risk score value, the top predictors that contributed to that score value are displayed, as shown in FIG. 9. The user may navigate along the trend plot backward in time to see how the patient's clinical data affected the risk score values, and drill down to those parameters along the trend plot over time. The user interface also provides a comprehensive listing of the patient's current and historic clinical data or variables, as shown in FIG. 10. For example, these clinical data may include risk score, GCS, body temperature, heart rate, respiration rate, blood pressure, SpO2 (pulse oximeter reading), arterial blood gases (BASE EXC ART), international normalized ratio (INR), white blood cell count (WBC), and red blood cell count (hemoglobin). Clicking on any variable causes the user interface to display the value for that factor or variable at a certain point in time within the context of a trend plot, as shown in FIG. 11.

The use of the tool IO described herein in the treatment of trauma patients reduces person-to-person variations in the composition of the care team and standardizes care of polytrauma patients. The use of the tool 10 also enables the care team to reduce their reliance on intuitive judgment, remove bias, and minimize experience-level induced differences in clinical results. This tool can be integrated directly into the clinical workflow and present a seamless experience to clinicians given the time-critical judgment windows that they face in the emergent/critical care setting. The tool 10 presents a customized and contextual drill-down user interface especially over time trends of physiological as well as other factors particular to the condition/use case like Trauma, in this case, to reduce most of the cognitive overload clinicians currently go through to if they were to themselves look for this information both within their EHR or log into any available separate dashboard. The latter are generally hosted on standalone technology platforms and sorely miss the much-needed context and rapid refresh cycle. Because this tool automatically ingests and presents information at of near real-time, the most recent updated information is always available to the care team members who are making time-critical life-saving decisions.

The electronic medical/health record (EMR or EHR) clinical data may be received from entities such as hospitals, clinics, pharmacies, laboratories, and health information exchanges, including: vital signs and other physiological data, data associated with comprehensive or focused history and physical exams by a clinician, nurse, or allied health professional; medical history; prior allergy and adverse medical reactions: family medical history; prior surgical history; emergency room and inpatient records; medication administration records; culture results; dictated clinical notes and records; gynecological and obstetric history; mental status examination; vaccination records; radiological imaging exams; invasive visualization procedures; psychiatric treatment history; prior histological specimens; laboratory data; genetic information; clinician's notes; networked devices and monitors (such as blood pressure devices and glucose meters); pharmaceutical and supplement intake information; and focused genotype testing. The EMR non-clinical data may include, for example, social, behavioral, lifestyle, and economic data; type and nature of employment; job history; medical insurance information; hospital utilization patterns; exercise information; addictive substance use; occupational chemical exposure; frequency of clinician or health system contact; location and frequency of habitation changes; predictive screening health questionnaires such as the patient health questionnaire (PHQ); personality tests; census and demographic data; neighborhood environments; diet; gender; marital status; education, proximity and number of family or care-giving assistants; address; housing status; and social media data. The non-clinical patient data may further include data entered by the patient, such as data entered or uploaded to a patient portal. Additional sources or devices of EMR data may provide, for example, lab results, medication assignments and changes, EKG results, radiology notes, daily weight readings, and daily blood sugar testing results. Additional non-clinical patient data may include, for example, community and religious organizational involvement; proximity and number of family or care-giving assistants; census tract location and census reported socioeconomic data for the tract; housing status; number of housing address changes; frequency of housing address changes, requirements for governmental living assistance; ability to make and keep medical appointments; independence on activities of daily living; hours of seeking medical assistance; location of seeking medical services; sensory impairments; cognitive impairments; mobility impairments; and economic status in absolute and relative terms to the local and national distributions of income; climate data; health registries, the number of family members, relationship status; individuals who might help care for a patient; and health and lifestyle preferences that could influence health outcomes. Certain data identified above are referred to as social determinants of health (SDOH) data that provide insight into the conditions in which people are born, grow, live, work and age, and may include factors like socioeconomic status, education level, neighborhood and physical environment, employment, and social support networks, as well as case of access to health care.

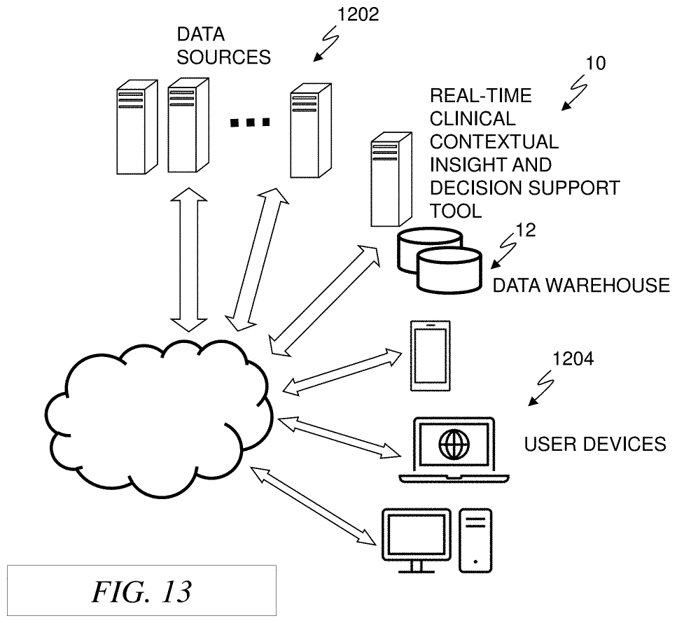

FIG. 13 is a simplified block diagram for the operational environment of the system and method IO described herein. The clinical insight and decision support tool IO can be hosted on a cloud-based platform (e.g., Azure) with cloud-based data warehouses 1200 that are configured to automatically access and receive patient clinical and non-clinical data sources 1202 via data pipeline, automated data flow, and real-time API as described above. Users may access the reporting and dashboard functions of the tool 10 via a variety of computing devices 1204, including, for example, mobile devices, laptop computers, notebook computers, notepads, and desktop computers. The cloud-based solution facilitates data replication, fault tolerance, and computational and data scalability without an on-premises infrastructure that requires enormous upfront investment. Further, load-balancing and database redundancy and mirroring mechanisms may be deployed to implement a fault-tolerant system.

The features of the present invention which are believed to be novel are set forth below with particularity in the appended claims. However, modifications, variations, and changes to the exemplary embodiments of the clinical contextual insight and decision support visualization tool described above will be apparent to those skilled in the art, and the system and method described herein thus encompasses such modifications, variations, and changes and are not limited to the specific embodiments described herein.

What is claimed is:

1. A clinical insight and decision support visualization tool, comprising:
a data ingestion logic module configured to automatically access, in real-time, electronic health data of a plurality of trauma patients being treated by a care team at a healthcare institution;
the data ingestion logic module further configured to automatically extract patient data from the electronic health data and process the extracted patient data for analysis;
a data analysis module configured to automatically apply at least one predictive model to analyze the processed patient data and determine a current mortality risk score value for each patient; and
a user interface module configured to present real-time and historical information for each trauma patient including a current mortality risk score value, a categorization of the current mortality risk score value, top contributors to the current mortality risk score value and their respective values, and a trend plot of historic mortality risk score values.

2. The clinical insight and decision support visualization tool of claim 1, wherein the data ingestion logic module is configured to extract patient data selected from the group consisting of age, Glasgow Coma Score (GCS), body temperature, heart rate, blood pressure, respiration rate, SpO2 (pulse oximeter reading), arterial blood gases (BASE EXC ART), white blood cell count (WBC), red blood cell count (hemoglobin), potassium level, creatinine, international normalized ratio (INR), and AST (aspartate aminotransferase indicative liver damage).

3. The clinical insight and decision support visualization tool of claim 1, wherein the user interface module is further configured to display a trend plot of the historic mortality risk score values wherein each plot data point is color-coded to indicate whether a data value is inside or outside of a desired value range.

4. The clinical insight and decision support visualization tool of claim 1, wherein the user interface module is further configured to display top contributors to each historic mortality risk score values and historic trend plots of top contributor values.

5. The clinical insight and decision support visualization tool of claim 1, wherein the user interface module is further configured to display a comprehensive list of all contributors used to determine each historic and current mortality risk score value.

6. The clinical insight and decision support visualization tool of claim 1, wherein the user interface module is further configured to display historic trend plots of top contributors to the current mortality risk score value.

7. The clinical insight and decision support visualization tool of claim 1, wherein the user interface module is further configured to display top contributors to a historic mortality risk data point on the trend plot when a pointer is placed over the historic mortality risk score data point.

8. The clinical insight and decision support visualization tool of claim 1, wherein the data analysis module operates during a time window having a configurable start time and end time measured from each patient's admission time.

9. A method for clinical insight and decision support visualization, the method comprising:
automatically accessing and receiving, in real-time, electronic health data of a plurality of trauma patients being treated by a care team at a healthcare institution;
automatically extracting electronic patient data from the received electronic health data and processing the extracted electronic patient data;
automatically applying at least one predictive model to the processed electronic patient data and automatically computing a current mortality risk score for each trauma patient; and
automatically presenting real-time and historical information related to each trauma patient to assist in treatment decision making by the care team, the presented information including a current mortality risk score value, a categorization of the current mortality risk score value, top contributors to the current mortality risk score value and their respective values, and a trend plot of historic mortality risk score values.

10. The clinical insight and decision support visualization method of claim 9, wherein automatically extracting electronic patient data comprises extracting patient data selected from the group consisting of age, Glasgow Coma Score (GCS), body temperature, heart rate, blood pressure, respiration rate, SpO2 (pulse oximeter reading), arterial blood gases (BASE EXC ART), white blood cell count (WBC), red blood cell count (hemoglobin), potassium level, creatinine, international normalized ratio (INR), and AST (aspartate aminotransferase indicative liver damage).

11. The clinical insight and decision support visualization method of claim 9, wherein automatically presenting historical information comprises displaying a trend plot of the historic mortality risk score values wherein each plot data point is color-coded to indicate whether a data value is inside or outside of a desired value range.

12. The clinical insight and decision support visualization method of claim 9, wherein automatically presenting historical information comprises displaying top contributors to each historic mortality risk score values and historic trend plots of top contributor values.

13. The clinical insight and decision support visualization method of claim 9, wherein automatically presenting historical information comprises displaying a comprehensive list of all contributors used to determine each historic and current mortality risk score value.

14. The clinical insight and decision support visualization method of claim 9, wherein the user interface module is further configured to display historic trend plots of top contributors to the current mortality risk score value.

15. The clinical insight and decision support visualization method of claim 9, wherein automatically presenting historical information comprises displaying top contributors to a historic mortality risk data point on the trend plot when a pointer is placed over the historic mortality risk score data point.

16. The clinical insight and decision support visualization method of claim 9, wherein automatically applying at least one predictive model comprises setting a time window for mortality risk score computation start time and end time measured from each patient's admission time.

17. A clinical insight and decision support visualization tool, comprising:
a data ingestion logic module configured to automatically interface with an electronic health data source and automatically access, in real-time, electronic health data associated with a plurality of patients being treated by a care team at a healthcare institution;
the data ingestion logic module further configured to automatically extract patient data from the electronic health data and process the extracted patient data for analysis;
a data analysis module configured to automatically apply at least one predictive model to analyze the processed patient data and determine a mortality risk score value based on the processed patient data for each patient at predetermined intervals;
a user interface module configured to present real-time and historical information for each patient including a current mortality risk score value, a categorization of the current mortality risk score value, top contributors to the current mortality risk score value and their respective values, and a trend plot of historic mortality risk score values; and
the user interface module being configured to present information selected from the group consisting of top contributors to each historic mortality risk score values and historic trend plots of top contributor values, historic trend plots of top contributors to the current mortality risk score value, and a comprehensive list of all contributors used to determine each historic and current mortality risk score value.

18. The clinical insight and decision support visualization tool of claim 17, wherein the data ingestion logic module is configured to extract patient data selected from the group consisting of age, Glasgow Coma Score (GCS), body temperature, heart rate, blood pressure, respiration rate, SpO2 (pulse oximeter reading), arterial blood gases (BASE EXC ART), white blood cell count (WBC), red blood cell count (hemoglobin), potassium level, creatinine, international normalized ratio (INR), and AST (aspartate aminotransferase indicative liver damage).

19. The clinical insight and decision support visualization tool of claim 17, wherein the user interface module is further configured to display a trend plot of the historic mortality risk score values wherein each plot data point is color-coded to indicate whether a data value is inside or outside of a desired value range.

20. The clinical insight and decision support visualization tool of claim 17, wherein the user interface module is further configured to display top contributors to a historic mortality risk data point on the trend plot when a pointer is placed over the historic mortality risk score data point.
